(19) United States
(12) Patent Application Publication
(10) Pub. No.: US 2002/0156536 A1
(43) Pub. Date: Oct. 24, 2002

(54) Polyethylene hip joint prosthesis with extended range of motion

(76) Inventors: William H. Harris, Belmont, MA (US); Orhun Muratoglu, Cambridge, MA (US); Murali Jasty, Weston, MA (US); Charles Bragdon, E. Weymouth, MA (US); Daniel O'Connor, E. Taunton, MA (US)

Correspondence Address: HELLER EHRMAN WHITE & MCAULIFFE LLP, 1666 K STREET,NW, SUITE 300, WASHINGTON, DC, 20006, US

(21) Appl. No.: 10/040,900

(22) Filed: Jan. 9, 2002

Related U.S. Application Data
(63) Continuation of application No. PCT/US99/16070, filed on Jul. 6, 1999.

(51) Int. Cl. A61F 2/32

(52) U.S. Cl. 623/22.17

(57) ABSTRACT

A hip joint prostheses including an acetabular cup (2) mounted in the hip socket (4) of the pelvis (6) is disclosed. The prosthesis also includes a head (8) which has a radius of curvature complementary to the cavity in the acetabular cup (2). The head (8) is typically made of metal. A neck (10) is connected to the head (8) joining the head (8) to the stem (12). The head (8), and the acetabular cup (2) are designed to allow a great deal of angular articulation. The bearing portions can be made with radiation treated ultrahigh molecular weight polyethylene polymer having substantially no detectable free radicals.

### CROSS REFERENCE TO RELATED APPLICATIONS

This is a continuation-in-part of U.S. Ser. No. 08/798,638, filed Feb. 11, 1997, which is a continuation-in-part of U.S. Ser. No. 08/726,313, filed Oct. 2, 1996, which is a continuation-in-part of U.S. Ser. No. 08/600,744, filed Feb. 13, 1996, now U.S. Pat. No. 5,879,400. The entire contents of each of these cases is incorporated herein by reference.

### FIELD OF THE INVENTION

This invention relates to hip joint prostheses.

### BACKGROUND OF THE INVENTION

Hip joint prostheses typically have a ball joint design that includes a cup-shaped bearing portion, called the acetabular cup, and a mating portion, which is typically a ball-shaped element, called the head. The head is articulated in the cavity of the cup to permit motion. In a full replacement hip joint prosthesis, the head is provided by removing the existing femur ball and implanting a prosthetic head with a rod-like member, known as the neck and stem, which is attached to the femur. In another design, known as a surface replacement prosthesis, the head is provided by resurfacing the existing femur ball with a covering, typically metal.

The cavity of the acetabular cup is typically defined by a layer of ultra-high molecular weight polyethylene polymer, called the polyethylene cup. The useful lifetime of the prosthesis is affected by wear of the polymer. One mechanism of wear is abrasion caused by the motion of the head. This abrasion can liberate fine particles which initiates biological processes ultimately leading to failure of the prosthesis. The failure mechanism is described further in U.S. Ser. No. 08/798,638, incorporated, supra.

The rate of wear is influenced by the size of the head and the thickness of the polyethylene cup. As the diameter of the head increases, the distance the head slides over the polyethylene cup for a given motion, known as distance travelled, also increases, which results in greater abrasional interaction between the head and polyethylene cup, which increases wear.

The polyethylene cup thickness affects wear rate because of contact stresses, which are related to the cushioning effect of the polyethylene cup when the head bears upon it. High contact stress increases wear. Contact stress increases as polyethylene cup thickness is reduced.

As a result of these phenomena, most standard hip joint prostheses using conventional polyethylene polymer cups have a head diameter of about 32 mm or less, typically about 22 mm or 28 mm, and polyethylene cup thicknesses of about 6 mm or more. While these dimensions can provide a reasonable prosthesis lifetime, e.g., a 10% failure rate in ten years, they can also compromise performance. For example, a small head diameter reduces the range of motion and can also increase the likelihood of dislocation.

A thick polyethylene cup restricts the head size, which may be a particular problem for patients with small pelvic sockets. The head size that may be used for a given pelvic socket size is limited by the thickness of the attachment mechanism for the acetabular cup, which may include bone cement and/or a metal shell, as well as the thickness of the polyethylene cup. For example, for patients with socket diameters of about 41 mm, the most common head size is only 22 mm. The small head size can limit the range of motion and increase the likelihood of dislocation compared to patients with larger sockets that can accommodate larger heads.

### SUMMARY OF THE INVENTION

This invention relates to polyethylene hip joint prostheses that have combinations of polyethylene cup thicknesses and head diameters that can extend range of motion and also have enhanced wear resistance. The range of motion can be extended by using one or a combination of a larger head size, a thin polyethylene cup, and non-hemispherical acetabular cup geometries. Wear resistance is enhanced by using an irradiated ultra-high molecular weight polyethylene polymer with substantially no detectable free radicals, a material discussed in U.S. Ser. No. 08/798,638. The modulus of elasticity of this polymer can also be selected to provide greater cushioning in a thinner polyethylene cup, which reduces contact stress and the likelihood of failure modes generally, and particularly in polyethylene cups with chamfered rims. In embodiments, for a given socket size, the polyethylene cup thickness is substantially reduced, which permits a substantially larger head, thus improving the range of motion and reducing the likelihood of dislocation. The lifetime of the prosthesis is extended by the wear resistance and lower modulus of the polyethylene. For example, for small socket diameters of about 41 mm, the head diameter may be about 28 mm or larger. This strategy has particular advantages for patients with small sockets, typical of the Asian population, for example, whose culture also involves deep flexion activities such as kneeling, e.g., in prayer, which requires extended motion range. For larger socket diameters, the invention permits head sizes that are much larger. For example, for a socket diameter of about 59 mm, the head diameter may be about 46 mm or more. The invention also permits prostheses of the surface replacement-type in which the existing ball of the femur is capped with a metal cup and the acetabulum is fitted with a thin cup of the polymer. In this case, the femur ball with the metal cup will typically have a diameter of 40 mm or more.

In one aspect, the invention features a hip joint prosthesis including a load bearing portion and a mating portion that define a cavity and a head articulated to provide motion such that θₘₐₓ is about 60° or more. At least one of the bearing portion and the mating portion include radiation treated ultra high molecular weight polyethylene polymer having substantially no detectable free radicals. The head cross-section is greater than about 35 mm, and the thickness of the polymer is about 1 mm to about 5 mm.

In another aspect, the invention features a hip joint prosthesis including a load bearing portion and a mating portion that define a cavity and a head articulated to provide motion. At least one of the bearing portion and the mating portion includes radiation treated ultra high molecular weight polyethylene polymer having substantially no detectable free radicals. The head cross-section is between about 20 mm to about 35 mm and the thickness of the polymer is about 1 mm to about 5 mm.

In another aspect, the invention features a hip joint prosthesis including a load bearing portion and a mating portion that define a cavity and a head articulated to provide motion. At least one of the bearing portion and the mating portion includes radiation treated ultra high molecular weight polyethylene having substantially no detectable free radicals. The thickness of the polymer is about 1 mm to about 2 mm.

In another aspect, the invention features a hip joint prosthesis including a load bearing portion and a mating portion that define a cavity and a head articulated to provide motion. At least one of the bearing portion and the mating portion includes radiation treated ultra high molecular weight polyethylene polymer having substantially no detectable free radicals. The head cross-section is greater than about 35 mm.

In still another aspect, the invention features a hip joint prosthesis system including: (a) a load bearing portion and a mating portion that define a cavity and a head articulated to provide motion, where at least one of the bearing portion and mating portion includes radiation treated ultra high molecular weight polyethylene; and (b) an attachment system for attaching the bearing portion to a patient. The attachment system includes bone cement, a metal shell, or a combination of bone cement and metal shell. The head cross-section (HS) satisfies the equation: HS=SS−2T_{C}−2T_{S}−2T_{L}, where SS is pelvic socket size, T_{C} is bone cement thickness, which is about 0 to about 6 mm, T_{S} is shell thickness, which is about 0 to about 5 mm, and T_{L} is polymer thickness which is about 1 mm to about 5 mm. When HS is greater than about 35 mm, θₘₐₓ is about 60° or greater. The invention also features a kit including this system and a method of implanting a hip joint prosthesis that includes determining socket size and implanting a prosthesis of this system.

Embodiments of the invention may include one or more of the following features. The angle θₘₐₓ can be about 60° to about 90°, or can be about 60° to about 70°. The head cross-section can be between about 35 mm and about 40 mm, or can be between about 40 mm and about 70 mm. The thickness of the polymer can be greater than about 2 mm to about 4 mm. The thickness of the polymer can also be about 3 mm, about 1 mm to about 2 mm, or about 1 mm to about 4 mm.

In addition, the bearing portion can have a rim chamfer, wherein the chamfer angle θ_{C} is substantially equal to θₘₐₓ. The polymer can have a storage modulus of about 850 MPa or less. The contact stress can be less than about 10 MPa.

The cavity depth of a prosthesis can be about 16 mm or more. In addition, the bearing portion can define a sphere segment cavity and the mating portion can be a ball head. The sphere segment can be a hemisphere, or the sphere segment can define less than a hemisphere in all directions of motion. For example, the sphere segment can define less than a hemisphere in a selected direction of motion and a hemisphere in another direction of motion. The bearing portion can include the polymer and the mating portion can include metal or ceramic. In addition, the mating portion can include a prosthetic ball member attached to the femur. The mating portion can include a shell covering an existing femoral ball.

The head cross-section of a prosthesis can be about 40 mm to about 70 mm, about 20 mm to about 35 mm, or about 35 mm to about 70 mm. The head size can be about 35 mm to about 70 mm. The head cross-section (HS) can be about 28 mm or more when the pelvic socket size (SS) is about 44 mm or less. Alternatively, the head cross-section can be about 32 mm or more when the pelvic socket size is about 43 mm or more, or the head cross-section can be is about 45 mm or more when the pelvic socket size is about 55 mm or more.

In the systems of the invention, T_{C} can be about 3 mm, T_{S} can be about 3.5 mm, and T_{L} can be about 3 to about 4 mm, for example about 3 mm. T_{L} can also be about 1 to about 2 mm.

Embodiments of the invention may have one or more of the following advantages. The prostheses can provide a range of motion approaching that of a natural biological joint. For example, the range of motion for the patient in the flexion/extension arc can be 120° or more, such as 120°-135°, which facilitates squatting, kneeling, bending over to tie a shoe, and the like.

As mentioned, these advantages can be beneficial in patients with small sockets because a thin polyethylene cup can accommodate a larger head. The extended range of motion and improved wear characteristics can also make practical prostheses for relatively young patients, e.g., younger than age 40, and those who have an active life-style demanding greater mobility. The improved wear reduces the frequency of prosthesis replacement, which minimizes the number of replacement procedures during a patient's lifetime, also an advantage for younger patients. The use of a thin polyethylene cup can also reduce the overall size of the cup-head combination, which provides greater flexibility in positioning the prosthesis within the socket. The improved stability of the larger heads against partial or full dislocation reduces the need for deepening of the inner diameter of the polyethylene cup with features such as countersinks, thus simplifying the prosthesis and the implant procedure and increasing the range of motion.

Still further aspects, features, and advantages follow.

### DESCRIPTION OF THE PREFERRED EMBODIMENTS

We first briefly describe the drawings.

### BRIEF DESCRIPTION OF THE DRAWINGS

FIG. 1 is a partial cross-sectional view of a hip joint prosthesis implanted in a patient;

FIG. 2 is a cross-sectional view of a hip joint prosthesis with a countersink;

FIG. 3 is a cross-sectional view of hip joint prosthesis having a thin polyethylene cup and a large head;

FIGS. 4 and 4a are expanded cross-sectional views of an polyethylene cup chamfer, illustrating the effect of increasing chamfer angle on chamfer or rim width;

FIG. 5 is a cross-sectional view of a hip joint prosthesis having a large head;

FIG. 6 is a cross-sectional view of hip joint prosthesis having an acetabular cup that defines less than a hemisphere;

FIG. 7 is a cross-sectional view of a prosthesis having a partially less than hemispherical cup;

FIG. 8 is a cross-sectional view of a surface-replacement hip joint prosthesis;

FIG. 9 is a cross-section though the pelvic socket illustrating selection of head size;

FIG. 10 is a plot of contact stress as a function of polyethylene cup thickness; and

FIG. 11 illustrates measurement of contact stress.

### DESCRIPTION

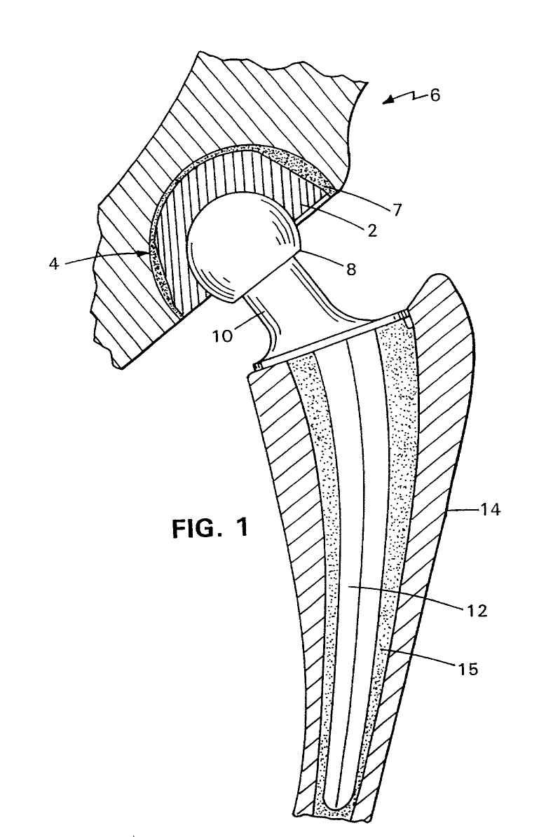

Referring to FIG. 1, a hip joint prosthesis includes an acetabular cup 2, which is mounted in the hip socket 4 of the pelvis 6. The prosthesis also includes a head 8 which has a radius of curvature complementary to the cavity in the acetabular cup. The head is typically made of metal, such as cobalt-chrome, or ceramic. A neck 10 is connected to the head. The neck 10 joins with a stem 12, which is connected to the femur 14 with a system 15 such as a press fit, a bone ingrowth surfacer, or cement. Alternatively, in a surface replacement prosthesis, the head could be the patient's existing femur ball, which is fitted with a metal or polymer cup.

The acetabular cup 2 is attached to the pelvis using an attachment system 7, which may include bone cement, a porous metal shell which permits bone ingrowth, or a combination of the cement and shell. Alternatively a friction fit attachment system may be used.

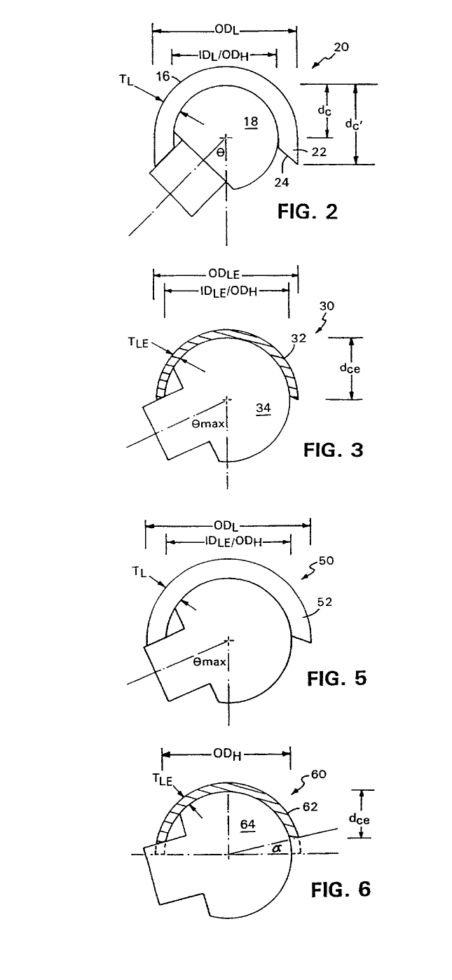

Referring as well to FIG. 2, a prosthesis has a polyethylene cup 16 made of ultrahigh molecular weight polyethylene of thickness T_{L}. The polyethylene cup 16 has an outer diameter OD_{L} and defines a hemispherical cavity with an internal diameter ID_{L}. A head 18 has, in the case of a hemispherical cup, a corresponding head cross-section diameter OD_{H}. The neck 20 is an elliptical or trapezoidal rod of metal or ceramic that has a maximum cross section of typically about 10 mm or more.

The polyethylene cup 16 also defines a cavity depth d_{C}, which in the case of a hemispherical polyethylene cup, corresponds to one half the inner diameter ID_{L}. To reduce the likelihood of dislocation, a countersink 22, is sometimes provided to increase the effective cavity depth to d_{C′}. The countersink is a cylindrical section of the polyethylene cup extending beyond the point at which the internal diameter of the polyethylene cup defines a hemisphere. The countersink has a chamfer 24 around its rim where the neck may engage it. The angle of the chamfer is selected in coordination with the head diameter and the neck size and geometry so that the chamfer is generally parallel with the neck at θₘₐₓ, the maximum angular motion of the prosthesis.

As evident in FIG. 2, the maximum angle θₘₐₓ is determined when the neck 20 attached to the head 18 engages a portion of the cup, in this example, the chamfer 24 on countersink 22. As a result, the maximum motion for the prosthesis is 2θ_{max.}

Referring to FIG. 3, an extended motion prosthesis 30 has a polyethylene cup 32 defining an outer diameter OD_{LE} which is the same as the polyethylene cup in FIG. 2, but has a much reduced thickness T_{LE}, thus providing a larger internal diameter ID_{LE}. The larger cavity provides a larger hemispherical cavity depth, d_{CE}, which reduces the likelihood of dislocation without the need for a countersink (although one may be optionally used), and accepts a larger head 34, of diameter OD_{H}, which increases the range of motion θₘₐₓ.

The polyethylene cup thickness is preferably about 1 mm to about 5 mm, or about 1 mm to about 2 mm, or about 3 mm to about 4 mm, most preferably around 3 mm. The head diameter may be larger than conventional heads or provide for a patient with a small socket, a head of conventional size but still larger than typical for a given attachment system. For large heads, the head diameter may be, e.g., greater than about 35 mm, preferably in the range of about 36 mm to about 70 mm, more preferably about 36 mm to about 40 mm or about 40 mm to about 70 mm. The cavity depth, d_{CE}, is preferably about 16 to about 40 mm. The maximum range of prosthesis motion, θₘₐₓ, is about 60° or greater, preferably about 60 to about 90°, preferably greater than about 62°, and more preferably from 60-70°. The angle θₘₐₓ provides a total possible range of motion in an arc of 2θₘₐₓ, which is preferably about 125° to about 135°.

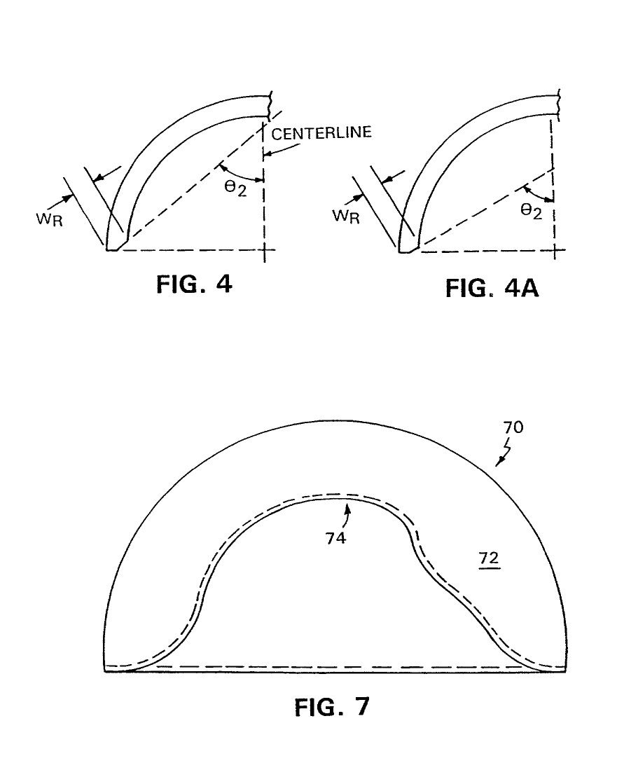

Referring to FIG. 4, the effect of extended θₘₐₓ of chamfer angle is illustrated. As mentioned, the chamfer 40 is the portion of the rim of the polyethylene cup that is beveled at an angle θ_{C} so that it is substantially parallel with the neck surface to support the neck when the prosthesis is at maximum extension. Chamfer angle θ_{C} is substantially equal to θₘₐₓ which provides a substantial material width, such as the rim width, wᵣ, so that the stress on the rim of the polyethylene cup is distributed over a wide rim region.

Referring to FIG. 4a, the chamfer angle θ_{C} increases as θₘₐₓ increases, producing greater force on the rim because the chamfer width decreases. As the thickness of the polyethylene cup decreases, force on the rim increases further still.

The prostheses described herein can utilize large heads and thin polyethylene cups because they employ highly wear resistant polyethylene material for the polyethylene cup, the head, or both. A wear resistant material permits a long prosthesis lifetime even under the extended distance-travelled effect of large heads. In addition, the wear effects, particularly at the chamfer and in thin layers of polyethylene, can be reduced by modifying the material so that it has a lower modulus of elasticity.

Wear resistant polyethylene materials that can be used in the prostheses described herein are discussed in U.S. Ser. No. 08/798,638, in WO 97/29793, and in U.S. Pat. No. 5,879,400. Briefly, the material is radiation treated ultra high molecular weight polyethylene having substantially no detectable free radicals. By substantially no detectable free radicals is meant substantially no free radicals as measured by electron paramagnetic resonance, as described in Jahan et al., J. Biomedical Materials Research 25:1005 (1991), the entire contents of which is incorporated herein by reference.

Free radicals include, e.g., unsaturated trans-vinylene free radicals. Ultra-high molecular weight polyethylene that has been irradiated below its melting point with ionizing radiation contains cross-links as well as long-lived trapped free radicals. These free radicals react with oxygen over the long-term and result in the embrittlement of the ultra-high molecular weight polyethylene through oxidative degradation. An advantage of the ultra-high molecular weight polyethylene and medical prostheses of this invention is that radiation treated ultra-high molecular weight polyethylene is used which has no detectable free radicals. The free radicals can be eliminated by any method which gives this result, e.g., by heating the ultra-high molecular weight polyethylene above its melting point such that substantially no residual crystalline structure remains. By eliminating the crystalline structure temporarily by melting, the free radicals are able to recombine and thus are eliminated. The ultra-high molecular weight polyethylene which is used in this invention has a cross-linked structure. An advantage of having a cross-linked structure is that it will reduce production of particles from the prosthesis during abrasion by the head.

For the prostheses described herein, this wear resistant polyethylene may also have a relatively low modulus of elasticity, which increases cushioning effect even in thin polyethylene cup, thus reducing contact stress generally, and particularly at the chamfer. Referring to FIG. 10, a plot of contact stress as a function of thickness illustrates that, for conventional polyethylene (UHMPE), contact stress increases quickly at small polyethylene cups thickness compared to the wear resistant radiation treated ultra-high molecular weight polyethylene, for which contact stresses are less at all levels. Preferably, for these prostheses, the storage modulus of elasticity is about 850 MPa or less, e.g. between about 100-800 MPa. The contact stress is preferably about 17 MPa for small sockets and 10 MPa for larger sockets. (Measurement of contact stress and storage modulus are discussed infra.)

The modulus of elasticity can be modified by varying the radiation treatment during manufacture of the polymer. Several techniques for manufacture of the polyethylene are provided in U.S. Ser. No. 08/798,638. These include cold irradiation and subsequent melting (CIR-SM), warm irradiation and subsequent melting (WIR-SM), warm irradiation adiabatic melting (WIR-AM or WIAM), and melt irradiation (MIR). Generally, in MIR, modulus of elasticity decreases with dose level. In WIR-AM and CIR-SM, after an initial decrease, the modulus is constant to about 15 Mrad but then declines at higher doses. Crystallinity level may be used as an indicator of modulus. Crystallinity as a function of dose is described in WO 97/29793 (see, e.g., FIG. 4).

Additional Embodiments

Referring now to FIG. 5, an extended motion prosthesis 50 has a polyethylene cup 52 of thickness T_{L} similar to conventional cup thickness, but defines a hemispherical cavity having an inner diameter of ID_{LE}, much larger than the conventional prosthesis to accept a large head of corresponding outer diameter. As evident, the larger head and greater cavity depth d_{CE} reduce the likelihood of dislocation without the need for an extension cylinder and increase range of motion θₘₐₓ. The polyethylene cups thickness in this case may be, e.g., about 6 to 8 mm. The ball diameter and range of motion may be as described above. As discussed above, the wear resistance of the irradiated ultra high molecular weight polyethylene having substantially detectable no free radicals withstands the distance travelled wear effect of the larger head.

Extended motion prostheses using large heads and/or thin polyethylene cups can also be implemented with non-hemispherical geometries. Referring to FIG. 6, a prosthesis 60 with a less than hemispherical polyethylene cup 62 is illustrated. The polyethylene cup 62 defines a large internal diameter ID_{L} to accommodate a large diameter head 64. The polyethylene cup does not extend to a full hemisphere but rather defines a sphere segment extending only to an angle α, defined between the center of the arc and the rim of the polyethylene cup. As evident, the sphere segment provides an extended motion compared to a hemisphere. In addition, the polyethylene cup provides a large cavity depth d_{CE} to reduce the likelihood of dislocation.

The relationship between the cavity depth and the angle α can be expressed as:

$${d}_{CE} = \frac{{ID}_{E}}{2} ( 1 - sin α )$$

The angle α is preferably between about 1-45°, more preferably between about 10-20°. The head diameter, polyethylene cup thickness, and cavity depths are preferably in the ranges given above. As discussed above, the wear resistance of the irradiated ultra high molecular weight polyethylene having substantially detectable no free radicals withstands the distance travelled wear effect of the larger head and thin polyethylene cups.

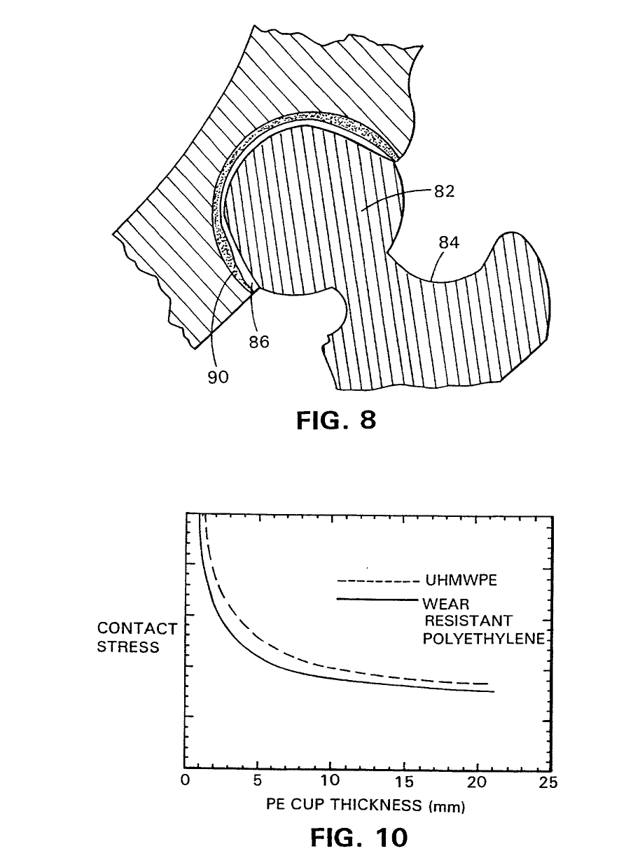

Referring to FIG. 7, in another embodiment, an extended motion prosthesis 70 has an polyethylene cup 72 that is non-hemispherical only in certain directions of motion. In this example, the polyethylene cup is substantially hemispherical in the direction of adductive motion, where a large range of motion does not normally occur, but is less than hemispherical in the direction of flexion/extension. The non-hemispherical portion 74 appears as a cut-out region in the body of the polyethylene cup. The angle of the cut out may be in the range of α, discussed above. The head diameter and polyethylene cup thickness are preferably in the ranges given above. As discussed above, the wear resistance of the irradiated ultra high molecular weight polyethylene having substantially detectable no free radicals withstands the distance travelled wear effect of the larger head.

Referring to FIG. 8, a thin polyethylene cup and large head can be used in surface replacement prostheses. In this case, the existing ball 82 on the femur 84 is covered with a femur cup 86 and the acetabulum is provided with a thin acetabular cup 90. The ball with the femur cup may be relatively large, with a diameter approaching or even exceeding the normal femur ball diameter. The acetabular cup and the ball cup are preferably thin, e.g. around 1 mm to 5 mm, preferably about 1 mm to about 2 mm, preferably about 3 mm. Either the acetabular cup or the ball cup may be formed of polymer, with the mating component made of metal or ceramic (e.g. 3 mm thick), or both cups may be polymer. The wear resistant polymer permits a large diameter ball and thin polymer layers without excessive wear.

In any of the embodiments, the thickness of the cup can also vary in the direction of different motions. For example, the cup may be thicker where greater wear is likely. Extended motion can still be achieved in spite of the thicker polyethylene cup by, e.g., implementing a less than hemispherical geometry or a much larger head hemispherical geometry.

In some embodiments, the head may also comprise the wear resistant polymer. The polymer may be provided as a thin covering or cup over a metal ball, or the entire ball may be made of polymer. In cases where the ball includes polymer, the acetabular cup may be metal, without a polymer cup.

The head is preferably spherical but may alternatively be nonspheroid, for example, the head may be ovaloid. The term head diameter or head size (HS) refers to the effective diameter determined by twice the radius of curvature of the head. For non-spheroid heads the cross section refers to the largest cross section.

Selection of Prostheses Parameters

The ultimate size of the head that may be implemented in a patient is determined in part by the method of attachment. Using a prosthesis with a thin polyethylene cup, as discussed above, can increase flexibility in terms of attachment technique because the overall diametric cross section of the acetabular cup and head combination will be reduced.

Generally, the prosthesis may be fixed to the patient's socket by several of known techniques, such as those using bone cement (e.g., methylmethacrylate), bone ingrowth, press-fit, screws, spikes, or a metal mesh embedded in polyethylene, as described, e.g., in Morscher et al., Clinical Orthopaedics and Related Research, No. 341, pp. 42-50 (1997). Metal shell and metal mesh systems may be used. The systems may be modular (e.g., the Trilogy System available from Zimmer, Warsaw, Ind.), in which case the components are implanted sequentially, or they may be a preassembled unit (e.g., the Sulmesh system, available from Sulzer Orthopedics, Baar, Switzerland).

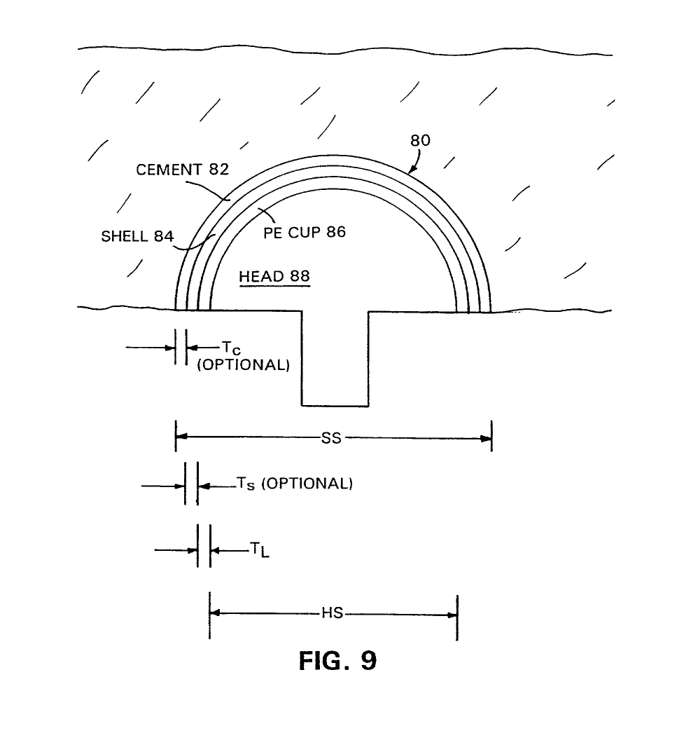

Referring to FIG. 9, the physician determines the size of the hip socket 80, e.g., by direct observation during surgery, and delivers the most appropriate attachment system. As illustrated, a socket size SS may be occupied by cement 82 of thickness T_{C}, a shell or mesh 84 of thickness T_{S}, a polyethylene cup 86 of thickness T_{L} and a head 88 of size HS. In some cases, no shell is used and in others, no cement is used. (Additionally a layer of cement between the cup and shell may also be used.)

The head size is calculated as follows:

HS=SS−2T_{C}−2T_{S}−2T_{L}

Preferably, the cup thickness may be about 1 mm to about 5 mm, most preferably about 3 mm. The shell or mesh thickness, when used, is about 1 mm to about 5 mm, preferably about 3 mm to about 4 mm. The cement thickness is about 1 mm to about 6 mm, typically 2-3 mm.

Table 1 illustrates examples of treatment of very small (41 mm), small (45 mm) and mid size (59 mm) socket sizes, using direct attachment of the polyethylene cup without cement.

TABLE I

| | Very Small | | |
|---|---|---|---|
| | Socket | Small Socket | Mid size Socket |
| SS | 41 mm | 45 mm | 59 mm |
| T_{S} | 3.5 mm | 3.5 mm | 3.5 mm |
| T_{L} | 3.0 mm | 3.0 mm | 3.0 mm |
| HS | 28 mm | 32 mm | 46 mm |

For a patient with a very small socket, the head size is 28 mm, for a patient with a small socket, the head size is 32 mm, and for a patient with a midsize socket, the head size is 46 mm.

Measurement of Storage Modulus

A dynamic mechanical analyzer is used to measure the storage (in-phase modulus) as a function of frequency and temperature. The control ultra-high molecular weight polyethylene (UHMWPE) used in this example was GUR 1050 ram extruded bar stock available from PolyHi Solidur, Ft. Wayne, Ind. Three test samples (˜3.2 mm wide; ˜1.3 mm thick; 25 mm long) were machined using a milling machine. The test samples were subsequently sterilized with gamma radiation in an oxygenless packaging.

The irradiated material was WIAM-TREATED GUR 1050 ram extruded bar stock. To prepare this material, polyethylene was preheated to 125° C., irradiated with a 10 MeV electron beam (Impela 10-50, E-Beam Services, Cranbury, N.J.) to a total dose level of 9.5 Mrad at a conveyor speed of 13.2 inches/minutes with a scan length of 32 inches. The samples were subsequently melt-annealed at 150° C. for two hours.

The test samples (˜3.2 mm wide; ˜1.3 mm thick; 25 mm long) were machined from the center of the irradiated hockey puck. The test samples were subsequently sterilized with ethylene oxide gas.

A Perkin Elmer Dynamic Mechanical Analyzer-7 (DMA-7) was used to measure the in-phase modulus of the control and WIAM-treated UHMWPE in 3-point bending. The DMA-7 was calibrated for the height, force, temperature, and furnace parameters following the instructions of the manufacturer. A reference material, epoxy of known modulus (−1.1 GPa), was used to validate the measured values of the in-phase modulus. The measured storage modulus of the reference epoxy is shown in the following table as a function of frequency.

TABLE II

| 1 Hz | 2 Hz | 3 Hz | 4 Hz | 5 Hz | 6 Hz | 7 Hz | 8 Hz | 9 Hz | 10 Hz |
|---|---|---|---|---|---|---|---|---|---|
| 1.105 | 1.105 | 1.108 | 1.106 | 1.109 | 1.107 | 1.111 | 1.111 | 1.111 | 1.108 |
| GPa | GPa | GPa | GPa | GPa | GPa | GPa | GPa | GPa | GPa |

Three test samples of each series were used to measure the in-phase and out-of-phase moduli at a temperature of 25° C. and at frequencies of 1 and 2 Hz. higher for both: 873±37 MPa for control and 676±30 MPa for WIAM.

TABLE III
Elastic modulus values measured for the control and WIAM treated UHMWPE.
The static load was 100 mN and the dynamic load was 80 mN.

| | | Storage | Loss | | | Static | Dynamic | Total | |
|---|---|---|---|---|---|---|---|---|---|
| | Frequency | Modulus | Modulus | | Phase | Stress | stress | stress | Amplitude |
| Sample ID | (Hz) | (MPa) | (MPa) | Tan (δ) | angle | (MPa) | (MPa) | (MPa) | (μm) |
| Control 1 | 1 | 832 | 64 | 0.07 | 4.4 | 0.53 | 0.42 | 0.95 | 26 |
| Control 2 | 1 | 830 | 59 | 0.07 | 4.2 | 0.35 | 0.38 | 0.73 | 14 |
| Control 3 | 1 | 905 | 64 | 0.07 | 4.0 | 0.35 | 0.28 | 0.63 | 13 |
| Average | | 855 ± 42 | 62 ± 2.9 | 0.07 | 4.2 ± 0.2 | | | | |
| Control 1 | 2 | 852 | 53 | 0.06 | 3.5 | 0.53 | 0.43 | 0.96 | 26 |
| Control 2 | 2 | 851 | 47 | 0.06 | 3.0 | 0.35 | 0.28 | 0.63 | 14 |
| Control 3 | 2 | 917 | 52 | 0.06 | 3.2 | 0.35 | 0.28 | 0.63 | 13 |
| Average | | 873 ± 37 | 51 ± 3.2 | 0.06 | 3.2 ± 0.5 | | | | |
| WIAM 1 | 1 | 653 | 69 | 0.1 | 6.1 | 0.56 | 0.44 | 1.00 | 37 |
| WIAM 2 | 1 | 623 | 64 | 0.1 | 5.9 | 0.33 | 0.26 | 0.59 | 16 |
| WIAM 3 | 1 | 695 | 62 | 0.1 | 5.1 | 0.36 | 0.29 | 0.65 | 17 |
| Average | | 657 ± 36 | 6.5 ± 3.6 | 0.1 | 5.7 ± 0.5 | | | | |
| WIAN 1 | 2 | 675 | 59 | 0.08 | 5.0 | 0.57 | 0.45 | 1.02 | 35 |
| WIAM 2 | 2 | 546 | 55 | 0.08 | 4.8 | 0.33 | 0.26 | 0.59 | 16 |
| WIAM 3 | 2 | 707 | 54 | 0.07 | 4.4 | 0.37 | 0.29 | 0.66 | 17 |
| Average | | 676 ± 30 | 56 ± 2.6 | 0.08 | 4.7 ± 0.3 | | | | |

Measurement of Contact Stress

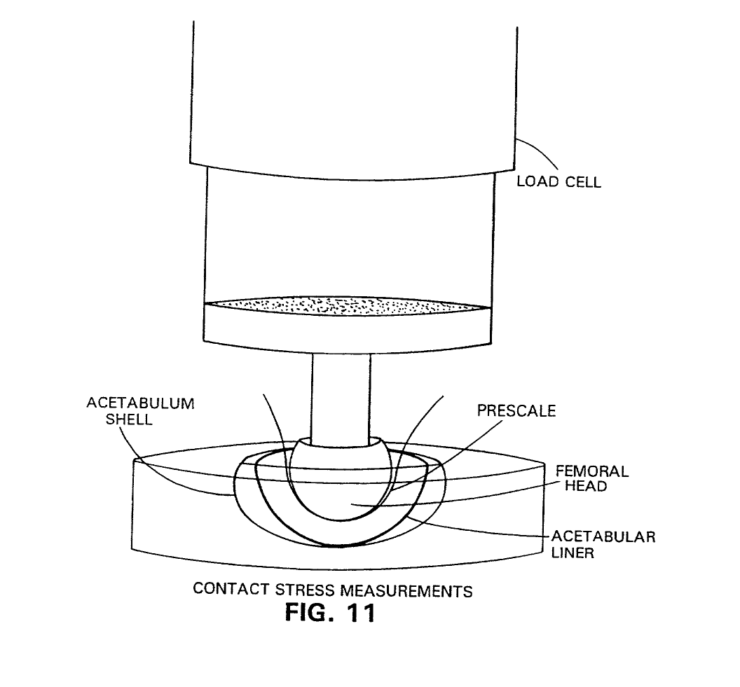

Referring to FIG. 11, contact stress is measured by observing color change in a stress sensitive film disposed between a head and a cup arranged in a hydraulic testing machine. The Fuji Prescale Film (Medium Mono Sheet Type film, available from Sensors Products, Inc., E. Hanover, N.J.) changes color under stress. The intensity of the color change on the film is proportional to the applied stress. A stress chart provided with the Fuji Prescale Film can then be used to determine the applied stress. An example of this measurement follows.

Fuji Film Prescale was used to quantify the contact stress between the cobalt-chrome femoral heads and control and WIAM-treated ultra-high molecular weight polyethylene liners. The Fuji film used was the medium pressure film with a stress range of 10-50 MPa (1422-7110 psi).

The following liners (i.e., polyethylene cups) were used to determine the contact stresses:

1. WIAM liners with 22 mm inner diameter and 39 mm outer diameter.

2. WIAM liners with 28 mm inner diameter and 49 mm outer diameter.

3. WIAM liners with 32 mm inner diameter and 55 mm outer diameter.

4. Control liners with 22 mm inner diameter and 39 mm outer diameter.

5. Control liners with 26 mm inner diameter and 49 mm outer diameter.

6. Control liners with 28 mm inner diameter and 49 mm outer diameter.

7. Control liners with 32 mm inner diameter and 55 mm outer diameter.

The WIAM liners used were made of DuraSul, available from Sulzer Orthopedics. The control liners used were InterOp acetabular liners, also available from Sulzer.

A 3 mm thin strip of Fuji Prescale Film was placed between the femoral head and the corresponding liner. The components were then loaded on an MTS servo hydraulic testing machine (MTS 810 Test System, available from MTS Systems Corp., Eden Prairie, Minn.) to a load of 2670N (600 lbs). Each load was applied for a duration of two minutes as recommended for the use of Fuji Prescale Film. The thin strip was then removed and the color change was analyzed using the stress chart provided with the fuji Prescale Film. The darkest region in each strip was analyzed with the color-coded stress chart. Therefore, the contact stress values reported here are the maximum encountered during loading. A total of three contact stress measurements were carried out for each homologous series. The contact stresses measured in each homologous series are listed in Table IV.

TABLE IV

| | Contact stress | Contact stress | Contact stress | Contact stress |
|---|---|---|---|---|
| | in 22 mm inner | in 26 mm inner | in 28 mm inner | in 32 mm inner |
| | diameter 39 mm | diameter XX mm | diameter 49 mm | diameter 55 mm |
| | outer diameter | outer diameter | outer diameter | outer diameter |
| | with 22 mm | with 26 mm | with 28 mm | with 32 mm |
| | femoral head | femoral head | femoral head | femoral head |
| Sample ID | (MPa) | (MPa) | (MPa) | (MPa) |
| Control 1 | 28 | 25 | 22 | 13 |
| Control 2 | 28 | 25 | 22 | 13 |
| Control 3 | 28 | 25 | 22 | 13 |
| Average | 28 ± 0 | 25 ± 0 | 22 ± 0 | 13 ± 0 |
| WIAM 1 | 26 | NA | 17 | 10 |
| WIAM 2 | 26 | NA | 17 | 10 |
| WIAM 3 | 26 | NA | 17 | 10 |
| Average | 26 ± 0 | NA | 17 ± 0 | 10 ± 0 |

As the results indicate, the contact stresses measured for the control liners were higher than those measured for WIAM-treated liners. Based on the contact stress values obtained from the other WIAM liners, it is believed that the contact stress in WIAM liners with 26 mm inner diameter and 49 mm outer diameter will be between 17 and 26 MPa. As discussed above, contact stress can be reduced by decreasing the modulus of elasticity.

Still further embodiments are within the following claims.

What is claimed is:

1. A hip joint prosthesis comprising a load bearing portion and a mating portion that define a cavity and a head articulated to provide motion such that θₘₐₓ is about 60° or more, wherein at least one of the bearing portion and the mating portion comprises radiation treated ultra high molecular weight polyethylene polymer having substantially no detectable free radicals, wherein the head cross-section is greater than about 35 mm, and where the thickness of said polymer is about 1 mm to about 5 mm.

2. The prosthesis of claim 1 wherein θₘₐₓ is about 60° to about 90°.

3. The prosthesis of claim 1 wherein θₘₐₓ is about 60° to about 70°.

4. The prosthesis of claim 1 wherein the head cross-section is between about 35 mm and about 40 mm.

5. The prosthesis of claim 1 wherein the head cross-section is about 40 mm to about 70 mm.

6. A hip joint prosthesis comprising a load bearing portion and a mating portion that define a cavity and a head articulated to provide motion, wherein at least one of the bearing portion and the mating portion comprises radiation treated ultra high molecular weight polyethylene polymer having substantially no detectable free radicals and wherein the head cross-section is between about 20 mm to about 35 mm and the thickness of said polymer is about 1 mm to about 5 mm.

7. The prosthesis of claim 1 or claim 6 wherein the thickness of the polymer is greater than about 2 mm to about 4 mm.

8. The prosthesis of claim 1 or claim 6 wherein the thickness is about 3 mm.

9. The prosthesis of claim 1 or claim 6 wherein the thickness is about 1 mm to about 2 mm.

10. The prosthesis of claim 1 or claim 6 wherein the bearing portion has a rim chamfer, wherein the chamfer angle θ_{C} is substantially equal to θₘₐₓ.

11. The prosthesis of claim 1 or claim 6 wherein the polymer has a storage modulus of about 850 MPa or less.

12. The prosthesis of claim 1 or claim 6 wherein the contact stress is less than about 10 MPa.

13. The prosthesis of claim 1 or claim 6 wherein the cavity depth is about 16 mm or more.

14. The prosthesis of claim 1 or claim 6 wherein the bearing portion defines a sphere segment cavity and said mating portion is a ball head.

15. The prosthesis of claim 14 wherein the sphere segment is a hemisphere.

16. The prosthesis of claim 14 wherein the sphere segment defines less than a hemisphere in all directions of motion.

17. The prosthesis of claim 14 wherein the sphere segment defines less than a hemisphere in a selected direction of motion and a hemisphere in another direction of motion.

18. The prosthesis of claim 14 wherein the bearing portion comprises said polymer and the mating portion comprises metal or ceramic.

19. The prosthesis of claim 14 wherein the mating portion comprises a prosthetic ball member attached to the femur.

20. The prosthesis of claim 14 wherein the mating portion comprises a shell covering an existing femoral ball.

21. A hip joint prosthesis comprising a load bearing portion and a mating portion that defines a cavity and a head articulated to provide motion, wherein at least one of the bearing portion and the mating portion comprises radiation treated ultra high molecular weight polyethylene having substantially no detectable free radicals and the thickness of the polymer is about 1 mm to about 2 mm.

22. The prosthesis of claim 21 wherein the head cross-section is about 40 mm to about 70 mm.

23. The prosthesis of claim 21 wherein the head cross-section is about 20 mm to about 35 mm.

24. A hip joint prosthesis comprising a load bearing portion and a mating portion that define a cavity and a head articulated to provide motion, wherein at least one of the bearing portion and the mating portion comprises radiation treated ultra high molecular weight polyethylene polymer having substantially no detectable free radicals and wherein the head cross-section is greater than about 35 mm.

25. The prosthesis of claim 24 wherein the head size is about 35 mm to about 70 mm.

26. A hip joint prosthesis system comprising:
(a) a load bearing portion and a mating portion that define a cavity and a head articulated to provide motion wherein at least one of said bearing portion and mating portion comprises radiation treated ultra high molecular weight polyethylene; and
(b) an attachment system for attaching said bearing portion to a patient, said attachment system comprising bone cement, a metal shell, or a combination of bone cement and metal shell,
wherein the head cross-section (HS) satisfies:
HS=SS−2T_{C}−2T_{S}−2T_{L}
where
SS is pelvic socket size,
T_{C} is bone cement thickness, which is 0 to about 6 mm,
T_{S} is shell thickness, which is 0 to about 5 mm,
T_{L} is polymer thickness which is about 1 mm to about 5 mm, and
when HS is greater than about 35 mm, θₘₐₓ is about 60° or greater.

27. The system of claim 26 wherein HS is about 28 30 mm or more when SS is about 44 mm or less.

28. The system of claim 26 wherein HS is about 32 mm or more when SS is about 43 mm or more.

29. The system of claim 26 wherein HS is about 45 mm or more when SS is about 55 mm or more.

30. The system of claim 26 wherein T_{C} is about 3 mm.

31. The system of claim 26 wherein T_{S} is about 3.5 mm.

32. The method of claim 26 wherein T_{L} is about 3 to about 4 mm.

33. The method of claim 26 wherein T_{L} is about 3 mm.

34. The method of claim 26 wherein T_{L} is about 1 to about 2 mm.

35. A kit comprising a prosthesis system described in claim 26.

36. A method of implanting a hip joint prosthesis, comprising determining socket size, and implanting a prosthesis described in claim 26.
